(19) United States
(12) Patent Application Publication
(10) Pub. No.: US 2016/0051179 A1
(43) Pub. Date: Feb. 25, 2016

(54) DETECTING AND MONITORING RECOVERY FROM TRAUMATIC BRAIN INJURY WITH PHOTOPLETHYSMOGRAPHY

(71) Applicant: Daniel Curtis Herman, Gainesville, FL (US)

(72) Inventor: Daniel Curtis Herman, Gainesville, FL (US)

(21) Appl. No.: 14/831,828

(22) Filed: Aug. 20, 2015

Related U.S. Application Data
(60) Provisional application No. 62/039,660, filed on Aug. 20, 2014.

(51) Int. Cl. A61B 5/00; A61B 5/0402; A61B 5/0205

(52) U.S. Cl. CPC A61B 5/4064; A61B 5/7278; A61B 5/7246; A61B 5/7282; A61B 5/0205; A61B 5/0402; A61B 5/6803; A61B 2503/10

(57) ABSTRACT

Systems and techniques for detecting and monitoring for traumatic brain injury and recovery are described. A system can include a photoplethysmography (PPG) system with a robust PPG sensor and a monitoring system with a detection and monitoring module. The detection and monitoring module, when executed, directs the monitoring system to: extract a pulse rate waveform from a signal from the PPG system containing information about a patient's heart; determine a pulse rate variability for the pulse rate waveform; and compare the pulse rate variability to a reference to determine a brain injury status.

### CROSS-REFERENCE TO RELATED APPLICATIONS

This application claims the benefit of U.S. Provisional Application No. 62/039,660 filed Aug. 20, 2014, the contents of which are incorporated herein by reference in their entirety.

### BACKGROUND

Photoplethysmography (PPG) refers to an optical technique for detecting blood volume changes in a tissue (e.g., skin). PPG uses one or more emitters to direct light at a tissue and one or more detectors to detect the light that is transmitted through the tissue (“transmissive PPG”) or the light that is reflected by the tissue (“reflectance PPG”). The volume of blood, or perfusion, of the tissue affects the amount of light that is transmitted or reflected. Thus, the PPG signal may vary with changes in the perfusion of the tissue.

Because the blood volume in a tissue changes with each heartbeat, the PPG signal also varies with each heartbeat, with a fundamental frequency typically around 1 Hz depending on heart rate. Traditionally, this component of the PPG signal is referred to as the “AC component” component of the signal, and is also often referred to as the “pulsatile component.” The AC component represents the increased light attenuation associated with the increase in microvascular blood volume with each heartbeat. In practice, the PPG waveform is often inverted.

Blood volume is also affected by other physiological processes in the body, including respiration, vasomotor activity and vasoconstrictor waves, Traube Hering Mayer (THM) waves, venous blood volume, thermoregulation, sympathetic and parasympathetic tone and certain pathologies. The changes in the PPG signal due to these and other physiological processes, along with changes in the PPG signal due to noise caused by non-physiological processes such as ambient light and bodily movement, have traditionally been referred to collectively as the “DC component.” With suitable electronic filtering and amplification both the AC and DC can be extracted for subsequent pulse wave analysis.

Concussions are notoriously difficult to diagnose and manage. Current best practice relies on subjective assessment of symptoms and brief neurocognitive screening tests. Symptoms may be masked by a patient to avoid detection of a concussion or return to activity sooner; conversely, symptoms may be amplified to avoid returning to work responsibilities. Neurocognitive screening tests have relatively low sensitivity and can be difficult to administer; furthermore, individuals may be able to “sandbag” baseline assessments (i.e., deliberately and/or deceptively perform slow or below one's ability during testing for ‘normal’ behavior) to make it easier to return to activity after a concussion.

### BRIEF SUMMARY

Pulse rate variability, extracted from a PPG signal, is presented as an objective measure of traumatic brain injury and recovery. Systems and techniques for detecting and monitoring recovery from traumatic brain injury with PPG are described.

In particular embodiments, a monitoring system can receive a signal from a PPG and/or ECG sensor, determine pulse rate variability from the signal, and assess traumatic brain injury (TBI) or recovery status based on the pulse rate variability.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

### BRIEF DESCRIPTION OF THE DRAWINGS

FIG. 1 illustrates an example operating environment in which detection and monitoring of recovery from traumatic brain injury can be accomplished.

FIG. 2 illustrates an example implementation of a process for detecting and monitoring recovery from traumatic brain injury.

FIGS. 3A and 3B illustrate components of example monitoring systems that may be used in certain implementations described herein.

### DETAILED DESCRIPTION

Systems and techniques for detecting and monitoring recovery from traumatic brain injury with (a) a PPG sensor that generates a PPG signal stream, and optionally (b) an ECG device that generates an electrocardiogram, are described. In some embodiments, provided are TBI sensing devices that are further configured to determine a PRV measurement from the electrocardiogram or PPG signal stream; to determine respiratory information from the PPG signal stream and/or a secondary respiration sensor; and to correct the PRV measurement by using the respiratory information from the PPG signal stream and/or another respiration sensor (e.g., thermistor, nasal airway pressure detector, etc.). Real-time evaluation and assessment of traumatic brain injury such as a concussion can be conducted by evaluating these signals for pulse rate variability.

As used herein, the term “pulse rate variability,” or PRV, will be used to refer to all measures of the variability in the cardiac cycle (length or rhythm), including, e.g., R to R variability and QT interval variability, and the term includes PRV data obtained from any source, including PPG and ECG (which is often referred to as heart rate variability or HRV but will be included in the general term PRV herein). As used herein, a “TBI” includes any injury to the brain sustained by an external force (directly or indirectly), including mild, moderate and severe TBIs. In particular embodiments, the TBI is a mild TBI, such as a concussion.

Systems and techniques are described that utilize pulse rate variability as an objective measure of traumatic brain injury. The PRV information from either the PPG or ECG signals may be analyzed by comparing with the respiratory information in any suitable manner, and when the PRV information is compared with the respiratory information, “corrected PRV information” may be obtained. The raw PRV information and/or the corrected PRV information may be analyzed to assess the degree or severity of traumatic brain injury.

The described devices and techniques can be used to determine whether a person has a concussion and/or time to return to play. Here, pulse rate variability can be used for (a) diagnosis of traumatic brain injury such as concussions, (b) monitoring clinical progression during recovery, and (c) determining prognosis and return to play (or work) based on their pulse rate variability normalizing over time.

The pulse rate variability can be compared to a baseline or a standard normal measure. The pulse rate variability can correlate to injury severity and symptomatic progression. The results can be used on their own or with respect to a previous injury. A baseline measure refers to a measure taken from a patient at a known state. A standard normal measure may be an average or normalized measure generated from one or more subjects. In some cases, the measures can include signals taken from rest, signals taken from various activities, or a combination thereof. In some cases, machine learning may be used to distinguish pulse rate variability occurring due to an activity from pulse rate variability relevant to injury severity and symptomatic progression.

A determination of pulse rate variability can be applied through standard sports equipment. In particular embodiments, the TBI sensing devices are used with or integral to a helmet, such as a sports helmet, or other head gear. In some embodiments, the helmet includes PPG sensor configured to obtain PPG measurements at the mastoid process, temporal, and/or supraorbital region. In particular embodiment, the TBI sensing devices can be integral to a mouthguard which includes PPG sensors configured to obtain measurements from the buccal mucosa. In particular embodiments, the TBI sensing devices can be integral to a uniform or other worn equipment such as shoulder pads or other sports equipment.

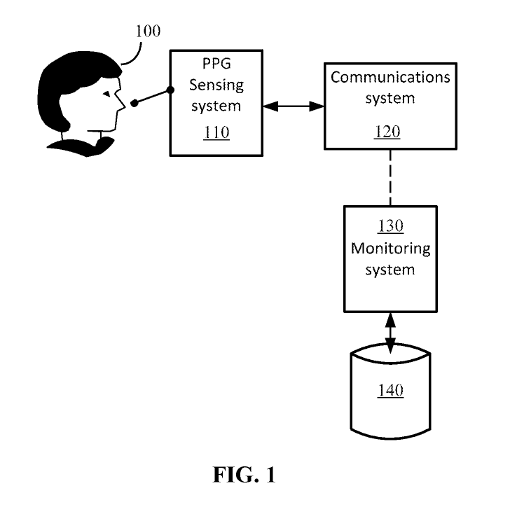

FIG. 1 illustrates an example operating environment in which detection and monitoring of recovery from traumatic brain injury can be accomplished. A subject 100, such as an athlete, can have his or her pulse rate variability sensed using a TBI sensing system 110. The sensing system 110 can include one or more photoplethysmograph sensors such as those designed for ear, nasal alae/septum or other highly perfused and responsive sites (e.g., buccal mucosa, forehead, mastoid process, etc.), and optionally at least one of an EKG sensor and another respiration sensor. For real-time monitoring during play, the sensing system 110 can be located at an appropriate region of a person's anatomy, for example by being embedded in a helmet or mouthguard.

Signals from the TBI sensing system 110 can be transmitted via a communications system 120 to a separate monitoring system 130. In some cases, monitoring system 130 may be at a remote location from the TBI sensing system 110; whereas in other cases, monitoring system 130 may be nearby and/or connected. The communication system 120 may include antennas, power amplifiers, RF circuitry, transceivers, and other communication circuitry. Communication of PPG and other signals by communications system 120 may be WiFi, cellular, radio/microwave frequency, ultra high frequency (UHF), or other far or near-field communication modality depending on implementation. In some cases, communication may be over wired (e.g., USB, HDMI) communication media.

The robust signal from a TBI sensing system using a PPG sensor directed to a highly perfused site can be processed to extract the pulse rate free from other physiologic signals, and can be corrected for respiratory rate, which is simultaneously recorded by the monitoring system 130. Once processed, the generated signal provides a highly accurate corrected pulse rate variability measure. Advantageously, because it is possible to correct for other physiologic signals and respiratory rate, the combined PPG sensing system 110 and monitoring system 130 can be highly resistant to motion artifact and may be easily applied with in situ, ambulatory, and hospital environments.

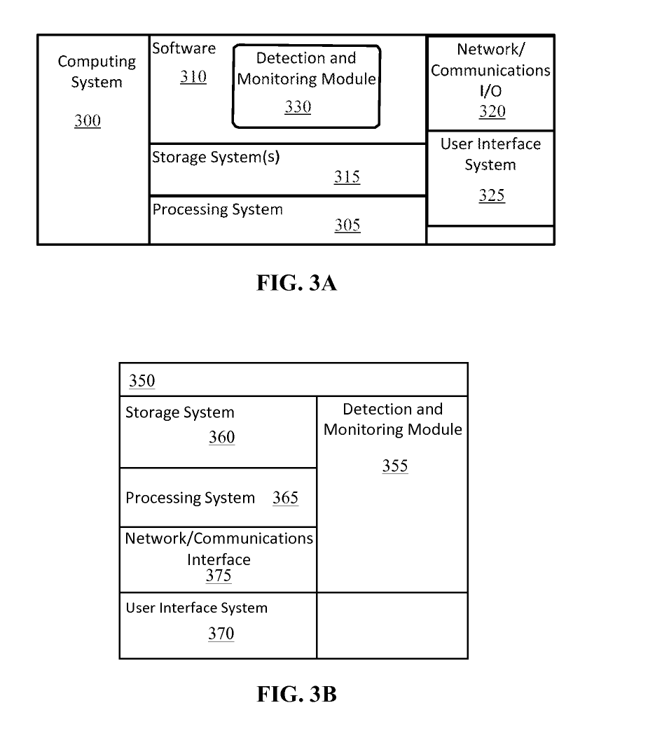

Monitoring system 130 can be a computing device (such as system 300 or system 350 described in more detail with respect to FIGS. 3A and 3B) that includes software, hardware, and/or firmware for analyzing the sensor signal to determine likelihood of injury and/or time to return to play. The software or hardware modules can include algorithms for various pulse rate variability measures.

Monitoring system 130 can be a handheld device or incorporated on a mobile phone, tablet computer, wearable computer, and the like (and which can be interfaced with the PPG sensing system 110). Monitoring system 130 may include or be in communication with (by wired or wireless means) storage media 140 on which baseline or reference measures may be stored for use by the monitoring system 130.

These systems may allow for real-time in-situ monitoring of pulse rate variability such that once a threshold was reached or a change threshold from baseline was reached, a notification would be generated (e.g. light, audible signal, page/text, etc.) that would alert healthcare providers or other officials as to the possibility that the monitored individual suffered a traumatic brain injury/concussion. These systems may also allow for comparisons to prior stored values of pulse rate variability and other clinical data when the individual is assessed in the field, subacutely, or in a clinical setting.

Values of pulse rate variability may also be recorded and tracked as an objective measure for determining injury resolution and allowing the individual back to play or in an occupational or military environment. The sensor of the device could be incorporated into sports, occupational, and/or military equipment, such as in a helmet (ear, mastoid process, forehead) and/or a mouthguard (buccal mucosa). Additionally, such a device can be interfaced and linked with a variety of additional systems, such as impact-sensing accelerometers, GPS systems, and thermistors for monitoring temperature.

As such, this device may be employed in real-time, in the immediate in-situ acute setting, the sub-acute clinical setting, and for management and return to activity clinical decision making.

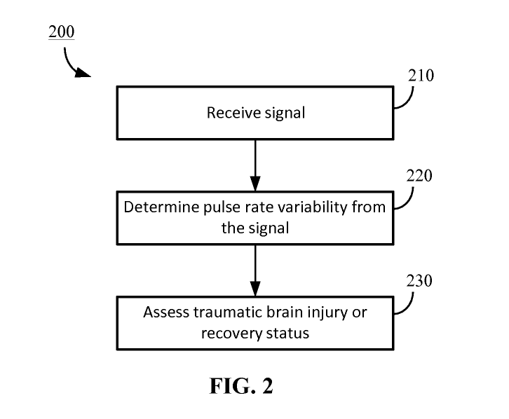

FIG. 2 illustrates an example implementation of a process for detecting and monitoring recovery from traumatic brain injury. Process 200 may be carried out by monitoring system 130. For example, a signal containing information about a patient's heart can be received by the system (210). This signal can be a PPG signal from a PPG sensor at or near a subject and communicated wired or wirelessly to the monitoring system. The pulse rate variability can be determined from the signal (220). In one implementation, the pulse rate waveform may be extracted from the signal and the pulse rate variability can be determined for the extracted pulse rate waveform. For example, the peaks in the pulse rate waveform can be determined and a linear analysis, a non-linear analysis, a frequency-based analysis, or a combination thereof with respect to the peaks in the pulse rate waveform can be performed to determine the pulse rate variability.

By using the pulse rate variability, traumatic brain injury or recovery status can be assessed (230). For example, the pulse rate variability can be compared to a reference to determine whether a patient has a concussion (concussed state), is in a normal state, is in recovery, or has some other brain injury status. An indication of the brain injury status can be provided, for example through a graphical user interface, as an alert or notification (via speaker, text, or some other output interface).

The PPG system and monitoring system and associated algorithms and software can be used in concussion screening, detection, and management in real-time, sideline/in-situ, or clinical settings.

The PPG-based system can provide a sensitive and objective measure that is resistant to “sandbagging” at baseline or masking/amplification after injury. Furthermore, this system can be employed in all environments (game or combat, sideline or in the field, clinical setting, etc.) and time points (real-time, acute, subacute, monitoring of progress and prognosis) relevant to concussion. Finally, application of the system may be non-invasive, very user-friendly, and of low cost.

Certain techniques set forth herein with respect to detecting and monitoring recovery from traumatic brain injury may be described in the general context of computer- executable instructions, such as program modules, executed by one or more computing devices. Generally, program modules include routines, programs, objects, components, and data structures that perform particular tasks or implement particular abstract data types.

Alternatively, or in addition, the functionality, methods and processes described herein can be implemented, at least in part, by one or more hardware modules (or logic components). For example, the hardware modules can include, but are not limited to, application-specific integrated circuit (ASIC) chips, field programmable gate arrays (FPGAs), system-on-a-chip (SoC) systems, complex programmable logic devices (CPLDs) and other programmable logic devices now known or later developed. When the hardware modules are activated, the hardware modules perform the functionality, methods and processes included within the hardware modules.

FIGS. 3A and 3B illustrate components of example monitoring systems that may be used in certain implementations described herein. Referring to FIG. 3A, monitoring system 300 may represent a computing device such as, but not limited to, a personal computer, a tablet computer, a reader, a mobile device, a personal digital assistant, a wearable computer, a smartphone, a tablet, a laptop computer (notebook or netbook), a gaming device or console, a desktop computer, a smart television or a dedicated monitoring device (hand-held or desktop). Accordingly, more or fewer elements described with respect to system 300 may be incorporated to implement a particular computing device. System 300 includes a processing system 305 of one or more processors to transform or manipulate data according to the instructions of software 310 stored on a storage system 315. Examples of processors of the processing system 305 include general purpose central processing units, application specific processors, and logic devices, as well as any other type of processing device, combinations, or variations thereof. The processing system 305 may be, or is included in, a system-on-chip (SoC) along with one or more other components such as network connectivity components (e.g., for network/communications interface 320), sensors, and video display components (e.g., for user interface system 325).

The software 310 can include an operating system (OS) and a detection and monitoring program module 330. Device operating systems generally control and coordinate the functions of the various components in the computing device, providing an easier way for applications to connect with lower level interfaces like the networking interface. Non-limiting examples of operating systems include Windows® from Microsoft Corp., Apple® iOS™ from Apple, Inc., Android® OS from Google, Inc., and the Ubuntu variety of the Linux OS from Canonical.

Storage system 315 may comprise any computer readable storage media readable by the processing system 305 and capable of storing software 310 including the detection and monitoring program module 330.

Storage system 315 may include volatile and nonvolatile, removable and non- removable media implemented in any method or technology for storage of information, such as computer readable instructions, data structures, program modules, or other data. Examples of storage media of storage system 315 include random access memory, read only memory, magnetic disks, optical disks, CDs, DVDs, flash memory, virtual memory and non-virtual memory, magnetic cassettes, magnetic tape, magnetic disk storage or other magnetic storage devices, or any other suitable storage media. In no case is the storage medium a propagated signal or carrier wave.

In addition to storage media, in some implementations, storage system 315 may also include communication media over which software may be communicated internally or externally. Storage system 315 may be implemented as a single storage device but may also be implemented across multiple storage devices or sub-systems co-located or distributed relative to each other. Storage system 315 may include additional elements, such as a controller, capable of communicating with processing system 305.

Software 310 may be implemented in program instructions and among other functions may, when executed by system 300 in general or processing system 305 in particular, direct system 300 or the one or more processors of processing system 305 to operate as described herein.

The system can further include user interface system 325, which may include input/output (I/O) devices and components that enable communication between a user and the system 300. User interface system 325 can include input devices such as a mouse, track pad, keyboard, a touch device for receiving a touch or stylus gesture from a user, a microphone for detecting speech, and other types of input devices and their associated processing elements capable of receiving user input. The user interface system 325 may also include output devices such as display screens, speakers, haptic devices for tactile feedback, and other types of output devices. In certain cases, the input and output devices may be combined in a single device, such as a touchscreen display which both depicts images and receives touch gesture input from the user. Visual output may be depicted on a display of the user interface system 325 in myriad ways, presenting graphical user interface elements, text, images, video, notifications, virtual buttons, virtual keyboards, or any other type of information capable of being depicted in visual form.

The user interface system 325 may also include user interface software and associated software (e.g., for graphics chips and input devices) executed by the OS in support of the various user input and output devices. The associated software assists the OS in communicating user interface hardware events to application programs using defined mechanisms. The user interface system 325 including user interface software may support a graphical user interface, a natural user interface, or any other type of user interface for accessing and/or interacting with information to/from the detection and monitoring program module 330.

Network/communications interface 320 may include communications connections and devices that allow for communication with other computing systems, sensor nodes and/or other devices over one or more communication networks. Examples of connections and devices that together allow for inter-system communication may include network interface cards, antennas, power amplifiers, RF circuitry, transceivers, and other communication circuitry. The connections and devices may communicate over communication media (such as metal, glass, air, or any other suitable communication media) to exchange communications with other computing systems or networks of systems. Transmissions to and from the network/communications interface 320 may be controlled by the OS, which informs applications, including the detection and monitoring module 330, of communications events when necessary.

Referring to FIG. 3B, monitoring system 350 may be or include a hardware detection and monitoring module 355. Detection and monitoring module 355 may include one or more of an ASIC, FPGA, SoC system, and CPLD. The monitoring system 350 can include storage system 360, processing system 365 (which may be separate from the detection and monitoring module 355 or embedded therein), user interface system 370, and network/communications interface 375.

Storage system 360 may include volatile and nonvolatile, removable and non- removable media implemented in any method or technology for storage of information, such as computer readable instructions, data structures, program modules, or other data. Examples of storage media of storage system 360 include random access memory, read only memory, magnetic disks, optical disks, CDs, DVDs, flash memory, virtual memory and non-virtual memory, magnetic cassettes, magnetic tape, magnetic disk storage or other magnetic storage devices, or any other suitable storage media. In no case is the storage medium a propagated signal or carrier wave. In addition to storage media, in some implementations, storage system 360 may also include communication media over which software may be communicated internally or externally.

User interface system 370 can include input devices such as a mouse, track pad, keyboard, a touch device for receiving a touch or stylus gesture from a user, a microphone for detecting speech, and other types of input devices and their associated processing elements capable of receiving user input. The user interface system 370 may also include output devices such as display screens, speakers, haptic devices for tactile feedback, and other types of output devices. In certain cases, the input and output devices may be combined in a single device, such as a touchscreen display which both depicts images and receives touch gesture input from the user. Visual output may be depicted on a display of the user interface system 370 in myriad ways, presenting graphical user interface elements, text, images, video, notifications, virtual buttons, virtual keyboards, or any other type of information capable of being depicted in visual form.

Network/communications interface 375 may include communications connections and devices that allow for communication with other computing systems, sensor nodes and/or other devices over one or more communication networks. Examples of connections and devices that together allow for inter-system communication may include network interface cards, antennas, power amplifiers, RF circuitry, transceivers, and other communication circuitry. The connections and devices may communicate over communication media (such as metal, glass, air, or any other suitable communication media) to exchange communications with other computing systems or networks of systems.

Certain embodiments of the described techniques for detecting and monitoring recovery from traumatic brain injury may be implemented as a computer process, a computing system, or as an article of manufacture, such as a computer program product or computer-readable medium. Certain methods and processes described herein can be embodied as software, code and/or data, which may be stored on one or more storage media. Certain embodiments of the invention contemplate the use of a machine in the form of a computer system within which a set of instructions, when executed, can cause the system to perform any one or more of the methodologies discussed above. Certain computer program products may be one or more computer-readable storage media readable by a computer system and encoding a computer program of instructions for executing a computer process.

Computer-readable media can be any available computer-readable storage media or communication media that can be accessed by the computer system. Communication media include the media by which a communication signal containing, for example, computer-readable instructions, data structures, program modules, or other data, is transmitted from one system to another system. The communication media can include guided transmission media, such as cables and wires (e.g., fiber optic, coaxial, and the like), and wireless (unguided transmission) media, such as acoustic, electromagnetic, RF, microwave and infrared, that can propagate energy waves. Although described with respect to communication media, carrier waves and other propagating signals that may contain data usable by a computer system are not considered computer-readable “storage media.”

By way of example, and not limitation, computer-readable storage media may include volatile and non-volatile, removable and non-removable media implemented in any method or technology for storage of information such as computer-readable instructions, data structures, program modules or other data. Examples of computer-readable storage media include volatile memory such as random access memories (RAM, DRAM, SRAM); non-volatile memory such as flash memory, various read-only-memories (ROM, PROM, EPROM, EEPROM), phase change memory, magnetic and ferromagnetic/ferroelectric memories (MRAM, FeRAM), and magnetic and optical storage devices (hard drives, magnetic tape, CDs, DVDs). As used herein, in no case does the term “storage media” consist of carrier waves or propagating signals.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

What is claimed is:

1. A method of detecting and monitoring for traumatic brain injury, comprising:
receiving a signal containing information about a patient's heart;
extracting a waveform from the signal;
determining a pulse rate variability from the waveform;
comparing the pulse rate variability to a reference to determine a brain injury status; and
providing an indication of the brain injury status.

2. The method of claim 1, wherein determining the pulse rate variability from the pulse rate waveform derived from a PPG signal comprises:
determining peaks in the pulse rate waveform; and
performing a linear analysis, a non-linear analysis, a frequency-based analysis, or a combination thereof with respect to the peaks in the pulse rate waveform to determine the pulse rate variability.

3. The method of claim 1, wherein the reference comprises a baseline measure received from the patient.

4. The method of claim 1, wherein the reference comprises a standard normalized measure from a set of control patients.

5. The method of claim 1, wherein the brain injury status comprises one of a recovery state, a concussed state, and a normal state.

6. A system for detecting and monitoring for traumatic brain injury, the system comprising:
a sensing system comprising at least one photoplethysmograph sensor, and
optionally one or more ECG sensors, one or more respiration sensors, or both, and
a monitoring system, in communication with the sensing system, that when executed:
extracts a pulse rate waveform from a signal from the sensing system containing information about a patient's heart;
determines a pulse rate variability for the pulse rate waveform;
corrects the pulse rate variability measurement by using the respiratory information from the PPG signal stream, the one or more respiration sensors or both; and
compares the pulse rate variability to a reference to determine a brain injury status.

7. The system of claim 6, further comprising standard sporting equipment selected from the group consisting of a helmet, mouthguard, uniform, or other wearable equipment wherein the standard sporting equipment houses the sensing system, the monitoring system, or both.

8. One or more computer readable storage media having instructions stored thereon that, when executed by a processing system, direct the processing system to:
extract a pulse rate waveform from a signal containing information about a patient's heart;
determine a pulse rate variability for the pulse rate waveform;
compare the pulse rate variability to a reference to determine a brain injury status; and
provide an indication of the brain injury status.
